(19) United States
(12) Patent Application Publication
(10) Pub. No.: US 2023/0201150 A1
(43) Pub. Date: Jun. 29, 2023

(54) THE COMBINATION OF ACETYL LEUCINE AND 4-AMINOPYRIDINE OR ACETAZOLAMIDE FOR TREATING ATAXIA

(71) Applicant: IntraBio Ltd., London (GB)

(72) Inventor: Michael STRUPP, Munich (DE)

(73) Assignee: IntraBio Ltd., London (GB)

(21) Appl. No.: 17/999,598

(22) PCT Filed: May 20, 2021
(86) PCT No.: PCT/IB2021/054399

§ 371 Date: Nov. 22, 2022

Related U.S. Application Data
(60) Provisional application No. 63/028,760, filed on May 22, 2020.

(51) Int. Cl. A61K 31/198; A61K 31/4409; A61K 31/433; A61P 25/14

(52) U.S. Cl. CPC A61K 31/198; A61K 31/4409; A61K 31/433; A61P 25/14

(57) ABSTRACT

The present disclosure provides methods of treating ataxia in a subject in need thereof by administering a combination of acetyl-leucine and 4-aminopyridine or a combination of acetyl-leucine and acetazolamide to the subject.

### BACKGROUND OF THE INVENTION

### Field of the Invention

The present disclosure provides a combination of acetyl-leucine and 4-aminopyridine or the combination of acetyl-leucine and acetazolamide for treating ataxia, e.g., episodic ataxia, in a subject.

### Background

Ataxias can be hereditary or acquired. Hereditary ataxias, a group of genetic disorders characterized by slowly progressive incoordination of gait often associated with poor coordination of hands, speech, and eye movements, and/or atrophy of the cerebellum, include autosomal dominant ataxias, e.g., spinocerebellar ataxias or episodic ataxias, and autosomal recessive ataxias, e.g., Niemann-Pick disease, gangliosidoses, or ataxia telangiectasia. Bird T D. Hereditary Ataxia Overview. 1998 Oct. 28 (Updated 2019 Jul. 25). In: Adam M P, Ardinger H H, Pagon R A, et al., editors. GeneReviews® (Internet). Seattle (Wash.): University of Washington, Seattle; 1993-2020. Acquired ataxias include sporadic ataxias or multiple system atrophies (MSA). Ashizawa and Xia, Continuum (Minneap Min) 22:1208-1226 (2016).

Episodic ataxias (EAs) are rare inherited neurological disorders characterized by recurrent episodes of a cerebellar ataxia with an imbalance of stance and gait, limb ataxia, dysarthria, and nystagmus. EAs are often triggered by physical or emotional stress, or alcohol, and are accompanied by nausea and vomiting. Kipfer and Strupp, Movement Disorders Clinical Practice 1:285-290, doi:10.1002/mdc3.12075 (2014); Jen and Wan, Handbook of Clinical Neurology 155:205-215, doi:10.1016/b978-0-444-64189-2.00013-5 (2018).

There are eight known subtypes of EA. Among them, EA1 and EA2 are clinically most relevant. EA2 has its onset typically in adolescence, but some cases with a late onset have been reported. Imbrici et al., Neurology 65:944-946, doi:10.1212/01.wnl.0000176069.64200.28 (2005). EA2 episodes generally last between minutes and hours, and are accompanied by migraine-like cephalgia in around 50% of patients. Jen et al., Neurology 62:17-22. doi:10.1212/01.wnl.0000101675.61074.50 (2004). EA2 patients frequently develop slowly progressive interictal baseline ataxia and distinct central ocular motor dysfunctions, e.g. mainly gaze evoked or downbeat nystagmus. Riant et al., Revue Neurologique 167:401-407, doi:10.1016/j.neurol.2010.10.016 (2011). EA2 is an inherited autosomal-dominant channelopathy, caused by mutations affecting the CACNA1A gene on chromosome 19p13, which encodes the alpha-1A subunit of the P/Q-type voltage-gated calcium channel (Cav2.1). Ophoff et al., Cell 87:543-552. doi:10.1016/s0092-8674(00)81373-2 (1996). Therapeutic principles in patients with EA include medical treatment and physiotherapy, occupational therapy for preserving gait function, and speech therapy. Ilg et al., Cerebellum (London, England) 13:248-268, doi:10.1007/s12311-013-0531-6 (2014); Gandini et al., J Neurol. 267:1211-1220, doi: 10.1007/s00415-020-09717-3 (2020).

There exists a need in the art for new medical therapies to treat ataxia in a subject.

### BRIEF SUMMARY OF THE INVENTION

Applicant has unexpectedly discovered that acetyl-leucine can be combined with 4-aminopyridine (4-AP) or acetazolamide to treat ataxia in a subject in need of such treatment, including, but not limited to, episodic ataxia (EA). In some embodiments, the subject has a deletion of cytosine and thymidine at position 2070-2071 in exon 16 of the CACNA1A gene.

In one aspect, the present disclosure provides acetyl-leucine in combination with 4-AP for use in the treatment of ataxia in a subject.

In one aspect, the present disclosure provides acetyl-leucine in combination with acetazolamide for use in the treatment of ataxia in a subject.

In another aspect, the present disclosure provides a method of treating ataxia in a subject in need thereof, the method comprising administering a combination of a therapeutically effective amount of acetyl-leucine and a therapeutically effective amount of 4-AP to the subject.

In another aspect, the present disclosure provides a method of treating ataxia in a subject in need thereof, the method comprising administering the combination of a therapeutically effective amount of acetyl-leucine and a therapeutically effective amount of acetazolamide to the subject.

In another aspect, the present disclosure provides the combination of acetyl-leucine and 4-AP for first line therapy to treat ataxia.

In another aspect, the present disclosure provides the combination of acetyl-leucine and acetazolamide for first line therapy to treat ataxia.

In another aspect, the present disclosure provides a kit comprising acetyl-leucine and 4-AP for treating ataxia in a subject.

In another aspect, the present disclosure provides a kit comprising acetyl-leucine and acetazolamide for treating ataxia in a subject.

It is to be understood that both the foregoing summary and the following detailed description are exemplary and explanatory only, and are not restrictive of the invention as claimed.

### DETAILED DESCRIPTION OF DRAWINGS

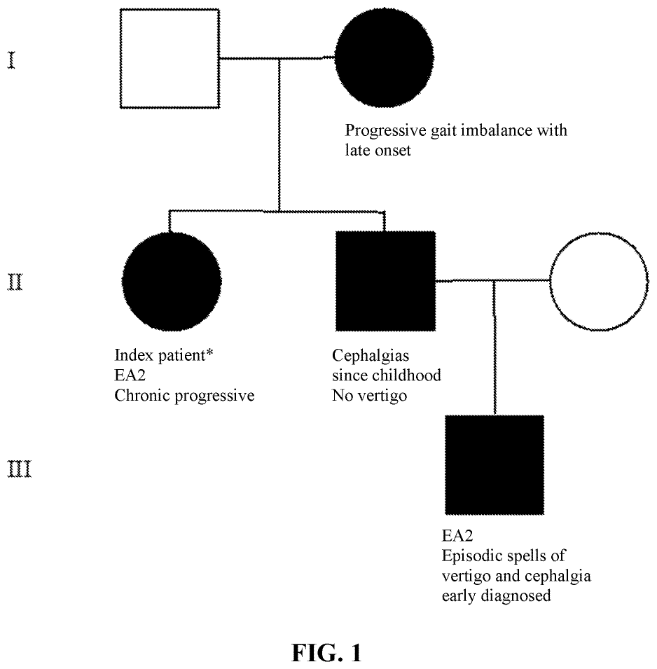

FIG. 1 is a pedigree chart of a German family carrying a newly identified heterozygous autosomal dominant CACNA1A mutation (NM_001127221.1: (c2070_2071delinsGGAG, p.(Phe690Leufs*9))). This mutation was found in four family members in three generations showing a heterogeneous phenotype. Legend: *=Index patient, square=male, circle=female, filled symbol=genetically affected subject, blank symbol=genetically unaffected subject.

### DETAILED DESCRIPTION OF THE INVENTION

In one embodiment, the present disclosure provides acetyl-leucine in combination with 4-AP or acetazolamide for use in treating ataxia in a subject. This is referred to as Embodiment 1.

The present disclosure provides particular Embodiments 2-23 as follows.

Embodiment 2. The combination for use according to Embodiment 1, wherein acetyl-leucine and 4-AP or acetazolamide are administered simultaneously.

Embodiment 3. The combination for use according to Embodiment 2, wherein acetyl-leucine and 4-AP or acetazolamide are administered as a single pharmaceutical formulation.

Embodiment 4. The combination for use according to Embodiment 2, wherein acetyl-leucine and 4-AP or acetazolamide are administered as two separate pharmaceutical formulations.

Embodiment 5. The combination for use according to Embodiment 1, wherein acetyl-leucine and 4-AP or acetazolamide are administered sequentially.

Embodiment 6. The combination for use according to Embodiment 5, wherein acetyl-leucine is administered to the subject before 4-AP or acetazolamide.

Embodiment 7. The combination for use according to Embodiment 5, wherein acetyl-leucine is administered to the subject after 4-AP or acetazolamide.

Embodiment 8. The combination for use according to any one of Embodiments 5-7, wherein acetyl-leucine and 4-AP or acetazolamide are administered about 1 minute to about 6 hours apart.

Embodiment 9. The combination for use according to Embodiment 8, wherein acetyl-leucine and 4-AP or acetazolamide are administered about 1 minute to 3 hours apart.

Embodiment 10. The combination for use according to Embodiment 9, wherein acetyl-leucine and 4-AP or acetazolamide are administered about 1 minute to 1 hour apart.

Embodiment 11. The combination for use according to any one of Embodiments 1-10, wherein acetyl-leucine and 4-AP or acetazolamide are administered orally.

Embodiment 12. The combination for use according to any one of Embodiments 1-11, wherein acetyl-leucine is administered once, twice, or three times per day.

Embodiment 13. The combination for use according to any one of Embodiments 1-12, wherein 4-AP or acetazolamide is administered once, twice, or three times per day.

Embodiment 14. The combination for use according to any one of Embodiments 1-13, wherein about 3 g to about 15 g of acetyl-leucine is administered per day.

Embodiment 15. The combination for use according to any one of Embodiments 1-14, wherein about 10 mg to about 30 mg of 4-AP or about 500 mg to about 1000 mg of acetazolamide is administered per day.

Embodiment 16. The combination for use according to any one of Embodiments 1-15, wherein acetyl-leucine and 4-AP or acetazolamide are administered as the first-line therapy to treat ataxia.

Embodiment 17. The combination for use according to any one of Embodiments 1-16, wherein the ataxia is EA.

Embodiment 18. The combination for use according to Embodiment 17, wherein the EA is episodic ataxia type 2 (EA2).

Embodiment 19. The combination for use of any one of Embodiments 1-18, wherein acetyl-leucine is combined with 4-AP.

Embodiment 20. The combination for use of any one of Embodiments 1-18, wherein acetyl-leucine is combined with acetazolamide.

Embodiment 21. The combination for use according to any one of Embodiments 1-20, wherein the acetyl-leucine is N-acetyl-DL-leucine.

Embodiment 22. The combination for use of any one of Embodiments 1-20, wherein the acetyl-leucine is N-acetyl-L-leucine.

Embodiment 23. The combination for use of any one of Embodiments 1-22, wherein the subject has a deletion of cytosine and thymidine at position 2070-2071 in exon 16 of the CACNA1A gene.

In another embodiment, the present disclosure provides a method of treating ataxia in a subject in need thereof, the method comprising administering a combination of a therapeutically effective amount of acetyl-leucine and (i) a therapeutically effective amount of 4-AP; or (ii) a therapeutically effective amount of acetazolamide, to the subject. This is referred to as Embodiment I.

The present disclosure provides particular Embodiments II-XXVI as follows.

Embodiment II. The method of Embodiment I, wherein acetyl-leucine and 4-AP or acetazolamide are administered simultaneously.

Embodiment III. The method of Embodiment II, wherein acetyl-leucine and 4-AP or acetazolamide are administered as a single pharmaceutical formulation.

Embodiment IV. The method of Embodiment II, wherein acetyl-leucine and 4-AP or acetazolamide are administered as two separate pharmaceutical formulations.

Embodiment V. The method of Embodiment I, wherein acetyl-leucine and 4-AP or acetazolamide are administered sequentially.

Embodiment VI. The method of Embodiment V, wherein acetyl-leucine is administered before 4-AP or acetazolamide.

Embodiment VII. The method of Embodiment V, wherein acetyl-leucine is administered after 4-AP or acetazolamide.

Embodiment VIII. The method of any one of Embodiments V-VII, wherein acetyl-leucine and 4-AP or acetazolamide are administered about 1 minute to about 6 hours apart.

Embodiment IX. The method of Embodiment VIII, wherein acetyl-leucine and 4-AP or acetazolamide are administered about 1 minute to 3 hours apart.

Embodiment X. The method of Embodiment IX, wherein acetyl-leucine and 4-AP or acetazolamide are administered about 1 minute to 1 hour apart.

Embodiment XI. The method of any one of Embodiments I-X, wherein acetyl-leucine and 4-AP or acetazolamide are administered orally.

Embodiment XII. The method of any one of Embodiments I-XI, wherein acetyl-leucine is administered once, twice, or three times per day.

Embodiment XIII The method of any one of Embodiments I-XII, wherein 4-AP or acetazolamide is administered once, twice, or three times per day.

Embodiment XIV. The method of any one of Embodiments I-XIII, wherein about 3 g to about 15 g of acetyl-leucine is administered per day.

Embodiment XV. The method of any one of Embodiments I-XIV, wherein about 10 mg to about 30 mg of 4-AP or about 500 mg to about 1000 mg of acetazolamide is administered per day.

Embodiment XVI. The method of any one of Embodiments I-XV, wherein acetyl-leucine and 4-AP or acetazolamide are administered as the first-line therapy to treat ataxia.

Embodiment XVII. The method of any one of Embodiments I-XVI, wherein the ataxia is EA.

Embodiment XVIII. The method of any one of Embodiments I-XVII, wherein the EA is episodic ataxia type 2 (EA2).

Embodiment XIX. The method of any one of Embodiments I-XVIII, wherein a therapeutically effective amount of acetyl-leucine is administered with a therapeutically effective amount of 4-AP.

Embodiment XX. The method of any one of Embodiments I-XIX, wherein a therapeutically effective amount of acetyl-leucine is administered with a therapeutically effective amount of acetazolamide.

Embodiment XXI. The method of any one of Embodiments I-XX, wherein the acetyl-leucine is N-acetyl-DL-leucine.

Embodiment XXII. The method of any one of Embodiments I-XX, wherein the acetyl-leucine is N-acetyl-L-leucine.

Embodiment XXIII. The method of any one of Embodiments I-XXII, wherein the subject has a deletion of cytosine and thymidine at position 2070-2071 in exon 16 of the CACNA1A gene.

Embodiment XXIV. A kit comprising acetyl-leucine and 4-AP or acetazolamide for treating ataxia in a subject.

Embodiment XXV. The kit of Embodiment XXIV further comprising instructions for administering the acetyl-leucine and 4-AP or acetazolamide to the subject.

Embodiment XXVI. The kit of embodiments XXIV or XXV, wherein the ataxia is episodic ataxia type 2.

### Definitions

As used herein, the singular forms “a,” “an,” and “the” include plural reference.

As used herein, the terms “approximately” and “about” should be generally understood to encompass ±20% of a specified amount, frequency or value. Numerical quantities given herein are approximate unless stated otherwise, meaning that the term “about” or “approximately” can be inferred when not expressly stated.

The terms “administer,” “administration,” or “administering” as used herein refer to (1) providing, giving, dosing and/or prescribing by either a health practitioner or his authorized agent or under his direction, the combination of acetyl-leucine and 4-AP or acetazolamide, and (2) putting into, taking or consuming by the subject or person himself or herself, acetyl-leucine and 4-AP or acetazolamide. Any reference to acetyl-leucine, 4-AP, or acetazolamide includes pharmaceutically acceptable salts of the same, even if not expressly stated.

A “pharmaceutically acceptable salt” as referred to herein, is any salt preparation that is appropriate for use in a pharmaceutical application. Pharmaceutically acceptable salts include, but are not limited to, amine salts, such as N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane and the like; alkali metal salts, such as lithium, potassium, sodium and the like; alkali earth metal salts, such as barium, calcium, magnesium and the like; transition metal salts, such as zinc, aluminum and the like; other metal salts, such as sodium hydrogen phosphate, disodium phosphate and the like; mineral acids, such as hydrochlorides, sulfates and the like; and salts of organic acids, such as acetates, lactates, malates, tartrates, citrates, ascorbates, succinates, butyrates, valerates, fumarates and the like.

Acetyl-leucine and 4-AP or acetazolamide may be formulated and administered to a subject in accordance with known teachings in the art. For example, acetyl-leucine and 4-AP or acetazolamide may be formulated as separate pharmaceutical compositions. These pharmaceutical compositions may comprise the active agent, i.e., acetyl-leucine or 4-AP or acetazolamide, and one or more pharmaceutically acceptable carriers. Acetyl-leucine and 4-AP or acetazolamide may also be formulated as single pharmaceutical composition comprising both active agents and one or more pharmaceutically acceptable carriers.

The pharmaceutical compositions comprising acetyl-leucine and 4-AP or acetazolamide, either separately or together in a single composition, may take any of a number of different forms depending on the manner in which they are to be used. Thus, for example, the pharmaceutical compositions may be in the form of a powder, tablet, capsule, liquid, ointment, cream, gel, hydrogel, aerosol, spray, micellar solution, transdermal patch, liposome suspension, or any other suitable form that may be administered to a subject in need of treatment.

A “pharmaceutically acceptable carrier” as referred to herein, is any known compound or combination of known compounds, e.g., excipients, carriers, etc., that are known to those skilled in the art to be useful in formulating pharmaceutical compositions. It will be appreciated that the carrier of the pharmaceutical composition should be one which is tolerated by the subject to whom it is given.

In one embodiment, the pharmaceutically acceptable carrier may be a solid, and the composition may be in the form of a powder or tablet. A solid pharmaceutically acceptable carrier may include, but is not limited to, one or more substances which may also act as flavouring agents, buffers, lubricants, stabilisers, solubilisers, suspending agents, wetting agents, emulsifiers, dyes, fillers, glidants, compression aids, inert binders, sweeteners, preservatives, dyes, coatings, or tablet-disintegrating agents. The carrier may also be an encapsulating material. In powders, the carrier may be a finely divided solid that is in admixture with the finely divided active agents according to the invention. In tablets, the active agent may be mixed with a carrier having the necessary compression properties in suitable proportions and compacted in the shape and size desired. The powders and tablets may, for example, contain up to 99% of the active agents. Suitable solid carriers include, for example, calcium phosphate, magnesium stearate, talc, sugars, lactose, dextrin, starch, gelatin, cellulose, polyvinylpyrrolidine, low melting waxes and ion exchange resins. In another embodiment, the pharmaceutically acceptable carrier may be a gel and the composition may be in the form of a cream or the like.

The carrier may include, but is not limited to, one or more excipients or diluents. Examples of such excipients are gelatin, gum arabicum, lactose, microcrystalline cellulose, starch, sodium starch glycolate, calcium hydrogen phosphate, magnesium stearate, talcum, colloidal silicon dioxide, and the like.

In another embodiment, the pharmaceutically acceptable carrier may be a liquid. In one embodiment, the pharmaceutical composition is in the form of a solution. Liquid carriers are used in preparing solutions, suspensions, emulsions, syrups, elixirs and pressurized compositions. Acetyl-leucine and/or 4-AP or acetazolamide may be dissolved or suspended in a pharmaceutically acceptable liquid carrier such as water, an organic solvent, a mixture of both or pharmaceutically acceptable oils or fats. The liquid carrier may contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid carriers for oral and parenteral administration include water (partially containing additives as above, e.g. cellulose derivatives, such as sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier may also be an oily ester such as ethyl oleate and isopropyl myristate. Sterile liquid carriers are useful in sterile liquid form compositions for parenteral administration. The liquid carrier for pressurised compositions may be a halogenated hydrocarbon or other pharmaceutically acceptable propellant.

Liquid pharmaceutical compositions, which are sterile solutions or suspensions, may be utilised by, for example, intramuscular, intrathecal, epidural, intraperitoneal, intravenous and subcutaneous injection. The active agent may be prepared as a sterile solid composition that may be dissolved or suspended at the time of administration using sterile water, saline, or other appropriate sterile injectable medium.

The compositions may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compositions may also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

In one embodiment, the pharmaceutical composition of acetyl-leucine, 4-AP, and acetazolamide is a solid oral dosage form, such as a tablet. In tablets, the active agent may be mixed with a vehicle, such as a pharmaceutically acceptable carrier, having the necessary compression properties in suitable proportions and compacted in the shape and size desired. The tablets may contain up to 99% by weight of the active agents.

Pharmaceutical compositions in solid oral dosage form, such as tablets, may be prepared by any method known in the art of pharmacy. Pharmaceutical compositions are usually prepared by mixing the active agent with conventional pharmaceutically acceptable carriers.

A tablet comprising acetyl-leucine may be formulated as is known in the art. Tanganil®, for example, includes wheat starch, pregelatinised maize (corn) starch, calcium carbonate and magnesium stearate as excipients. The same, or similar, excipients, for example, may be employed with the present disclosure.

The composition of each 700 mg Tanganil® tablet is as follows: 500 mg acetyl-DL-leucine, 88 mg wheat starch, 88 mg pregelatinised maize (corn) starch, 13 mg calcium carbonate and 11 mg magnesium stearate. The same tablets, for example, may be employed in the methods of the present disclosure.

As discussed above, acetyl-leucine and 4-AP or acetazolamide may be formulated and administered as a pharmaceutical composition taking any number of different forms. For example, acetyl-leucine may be formulated as a pharmaceutical composition to facilitate its delivery across the blood-brain barrier. As a further example, acetyl-leucine may be formulated as a pharmaceutical composition for bypassing the blood-brain barrier.

In one embodiment, the pharmaceutical composition, e.g., comprising acetyl-L-leucine, or salt thereof, is formulated for nanodelivery, e.g., colloidal drug-carrier systems. Suitable examples include but are not limited to liposomes, nanoparticles (e.g., polymeric, lipid and inorganic nanoparticles), nanogels, dendrimers, micelles, nanoemulsions, polymersomes, exosomes, and quantum dots. See, e.g., Patel et al., “Crossing the Blood-Brain Barrier: Recent Advances in Drug Delivery to the Brain,” CNS Drugs 31:109-133 (2017); Kabanov et al., “New Technologies for Drug Delivery across the Blood Brain Barrier,” Curr Pharm Des., 10(12):1355-1363 (2004); Cheng et al., “Highly Stabilized Curcumin Nanoparticles Tested in an In Vitro Blood-Brain Barrier Model and in Alzheimer's Disease Tg2576 Mice,” The AAPS Journal, vol. 15, no. 2, pp. 324-336 (2013); Lähde et al. “Production of L-Leucine Nanoparticles under Various Conditions Using an Aerosol Flow Reactor Method,” Journal of Nanomaterials, vol. 2008, article ID 680897 (2008).

In one embodiment, the pharmaceutical composition, e.g., comprising N-acetyl-L-leucine, or salt thereof, is formulated for direct delivery to the central nervous system (CNS), such as by injection or infusion. Formulations for and methods of direct delivery to the CNS are known in the art. See, e.g., U.S. Pat. No. 9,283,181. Examples of such administration include but are not limited to intranasal, intraventricular, intrathecal, intracranial, and delivery via nasal mucosal grafting. In one embodiment, the pharmaceutical composition is administered by intracerebroventricular infusion.

In one embodiment, the pharmaceutical composition is formulated for (and administered by) intranasal delivery. See, e.g., Hanson et al., “Intranasal delivery bypasses the blood-brain barrier to target therapeutic agents to the central nervous system and treat neurodegenerative disease,” BMC Neurosci. 9(Suppl 3): S5 (2008). In one embodiment, the pharmaceutical composition is formulated for (and administered by) delivery via a nasal mucosal graft. In one embodiment, the pharmaceutical composition is formulated for (and administered by) intracerebroventricular injection or infusion. In another embodiment, the pharmaceutical composition is formulated for (and administered by) intrathecal intracisternal injection or infusion. In one embodiment, the pharmaceutical composition is formulated for (and administered by) intrathecal lumbar injection or infusion.

Various techniques may be used including, without limitation, injection through a burrhole or cisternal or lumbar puncture or the like as known in the art. Various devices, whether internal (e.g., implanted) or external, may be used for delivery as known in the art, such as pumps, catheters, reservoirs, etc. In one embodiment, the administration interval is once every two weeks.

The term “ataxia” refers to impaired coordination of voluntary muscle movement in a subject. Ataxias can be hereditary or acquired. Hereditary ataxias, a group of genetic disorders characterized by slowly progressive incoordination of gait often associated with poor coordination of hands, speech, and eye movements, and/or atrophy of the cerebellum, include autosomal dominant ataxias, e.g., spinocerebellar ataxias or episodic ataxias, and autosomal recessive ataxias, e.g., Niemann-Pick disease, gangliosidoses, or ataxia telangiectasia. Bird T D. Hereditary Ataxia Overview. 1998 Oct. 28 (Updated 2019 Jul. 25). In: Adam M P, Ardinger H H, Pagon R A, et al., editors. GeneReviews® (Internet). Seattle (Wash.): University of Washington, Seattle; 1993-2020; Beaudin et al., 4:3 https://doi.org/10.1186/s40673-017-0061-y (2017). Acquired ataxias include sporadic ataxias or multiple system atrophies (MSA). Ashizawa and Xia, Continuum (Minneap Min) 22:1208-1226 (2016). In one embodiment, the ataxia is episodic ataxia, e.g. episodic ataxia type 1 to 7. In another embodiment, the episodic ataxia is episodic ataxia type 1 or type 2. In another embodiment, the episodic ataxia is episodic ataxia type 2. In another embodiment, the subject has a mutation in the CACNA1A gene, which such mutation can include, but is not limited to, one or more of those described in Sintas et al., Sci Rep 7:2514 doi: 10.1038/s41598-017-02554-x (2017). In another embodiment, the subject has a deletion of cytosine and thymidine at position 2070-2071 in exon 16 of the CACNA1A gene. Kim et al., J Clin Neurol 2:268-271 (2006); Denier et al., Neurology; 52:1816-1821 (1999).

The term “episodic ataxia” or “EA” refers to a disorder characterized by recurrent spells of truncal ataxia and incoordination that last minutes to hours. Eight subtypes of episodic ataxia have been defined according to clinical and genetic characteristics. episodic ataxia type 2 (EA2) is the most common subtype. EA2 episodes are characterized by recurrent ataxia, slurred speech for several hours, and interictal nystagmus. The onset is typically early in life, but patients with a much later onset have also been reported. Vertigo and fluctuating general weakness are common. Other symptoms include, but are not limited to, dysarthria, diplopia, tonic upward gaze, headache, seizure, dystonia, and/or cognitive impairment. Choi and Choi, J Mov Disord 9:129-135, DOI: https://doi.org/10.14802/jmd.16028 (2016).

The term “acetyl-leucine” refers collectively to N-acetyl-DL-leucine (ADLL), or a pharmaceutically acceptable salt thereof; N-acetyl-D-leucine (ADL), or a pharmaceutically acceptable salt thereof; and N-acetyl-L-leucine (ALL), or a pharmaceutically acceptable salt thereof. The term acetyl-leucine includes isotopically-labelled analogs of N-acetyl-DL-leucine, N-acetyl-D-leucine, and N-acetyl-L-leucine, wherein one or more atoms are replaced by an atom having a different atomic mass or mass number. Examples of isotopes that can be incorporated include isotopes of hydrogen, carbon, nitrogen, and oxygen, such as ²H (or deuterium (D)), ³H, ¹¹C, ¹³C, ¹⁴C, ¹⁵N, ¹⁸O, and ¹⁷O. In one embodiment, provided is an isotopically-labelled analog of acetyl-leucine, wherein substantially all of the atoms at a position within acetyl-leucine are replaced by an atom having a different atomic mass or mass number. In another embodiment, provided is an isotopically-labelled analog of acetyl-leucine, wherein a portion of the atoms at a position within acetyl-leucine are replaced, e.g., acetyl-leucine is enriched at one or more positions with an atom having a different atomic mass or mass number. Isotopically-labelled acetyl-leucine can be prepared by methods known in the art.

In one embodiment, the N-acetyl-DL-leucine, N-acetyl-D-leucine, or N-acetyl-L-leucine is not isotopically-labelled.

In one embodiment, the isotopically-labelled analog is a deuterated analog of N-acetyl-DL-leucine, N-acetyl-D-leucine, or N-acetyl-L-leucine, wherein one or more hydrogen atoms are replaced with deuterium. In one embodiment, one hydrogen atom of N-acetyl-DL-leucine, N-acetyl-D-leucine, or N-acetyl-L-leucine is replaced with deuterium. In another embodiment, two hydrogen atoms of N-acetyl-DL-leucine, N-acetyl-D-leucine, or N-acetyl-L-leucine are replaced with deuterium. In another embodiment, three hydrogen atoms of N-acetyl-DL-leucine, N-acetyl-D-leucine, or N-acetyl-L-leucine are replaced with deuterium. In another embodiment, four hydrogen atoms of N-acetyl-DL-leucine, N-acetyl-D-leucine, or N-acetyl-L-leucine are replaced with deuterium. In another embodiment, five hydrogen atoms of N-acetyl-DL-leucine, N-acetyl-D-leucine, or N-acetyl-L-leucine are replaced with deuterium. In another embodiment, six hydrogen atoms of N-acetyl-DL-leucine, N-acetyl-D-leucine, or N-acetyl-L-leucine are replaced with deuterium.

In one embodiment, the acetyl-leucine used in the methods of the present disclosure is N-acetyl-DL-leucine, or a deuterated analog thereof. In another embodiment, the acetyl-leucine used in the methods of the present disclosure is N-acetyl-D-leucine, or a deuterated analog thereof. In another embodiment, the acetyl-leucine used in the methods of the present disclosure is N-acetyl-L-leucine, or a deuterated analog thereof.

In another embodiment, the acetyl-leucine used in the methods of the present disclosure is N-acetyl-DL-leucine. In another embodiment, the acetyl-leucine used in the methods of the present disclosure is N-acetyl-D-leucine. In another embodiment, the acetyl-leucine used in the methods of the present disclosure is N-acetyl-L-leucine.

The terms “4-aminopyridine,” “4-AP,” or “4AP” refer to the drug also known as fampridine, dalfampridine, or Ampyra®. The chemical structure of 4-AP is:

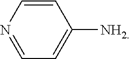

The term “acetazolamide” refers to the drug also known as Diamox. The chemical structure of acetazolamide is:

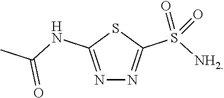

“Administered in combination” and similar phrases mean that two agents, e.g., acetyl-DL-leucine, acetyl-D-leucine, or acetyl-L-leucine; and (i) 4-AP; or (ii) acetazolamide are administered concurrently to the subject being treated. In one embodiment, acetyl-DL-leucine, acetyl-D-leucine, or acetyl-L-leucine and 4-AP, or acetyl-DL-leucine, acetyl-D-leucine, or acetyl-L-leucine and acetazolamide are administered in combination as a first line therapy to treat EA, e.g., EA2. In some embodiments, the subject does not respond to treatment with 4-AP as a single agent, and/or experiences unwanted side effects. In some embodiments, the subject does not respond to treatment with acetazolamide as a single agent, and/or experiences unwanted side effects.

“First line therapy” means a treatment regimen generally accepted or recommended by the medical establishment or a regulatory authority, e.g., the U.S. Food and Drug Administration or the European Medicines Agency, for the initial treatment of a condition, disease, or disorder.

“Concurrently” means that each active agent is administered either (i) simultaneously; or (ii) sequentially in any order at different points in time. A combination of two agents is considered to be administered simultaneously if each agent is administered to the subject less than 1 minute apart. If not administered simultaneously, it is meant that both agents are administered to a subject in a sequence and sufficiently close in time so as to provide the desired therapeutic effect and can act in concert to treat EA.

In one embodiment, acetyl-leucine and 4-AP or acetazolamide are administered separately, in any appropriate form and by any suitable route. In one embodiment, both acetyl-leucine and 4-AP or acetazolamide are orally administered to the subject as tablets or capsules.

In one embodiment, acetyl-leucine is administered to the subject 1 minute to 24 hours before the administration of 4-AP or acetazolamide to the subject. For example, acetyl-leucine is administered 5 minutes, 10 minutes, 15 minutes, 30 minutes, 45 minutes, 1 hour, 2 hours, 4 hours, 6 hours, or 12 hours before the administration of 4-AP or acetazolamide to a subject.

In another embodiment, acetyl-leucine is administered simultaneously with 4-AP or acetazolamide to the subject.

In another embodiment, acetyl-leucine is administered to the subject 1 minute to 24 hours after the administration of 4-AP or acetazolamide to the subject. For example, acetyl-leucine is administered 5 minutes, 10 minutes, 15 minutes, 30 minutes, 45 minutes, 1 hour, 2 hours, 4 hours, 6 hours, or 12 hours after, the administration of 4-AP or acetazolamide to a subject.

In another embodiment, acetyl-leucine and 4-AP or acetazolamide are administered to the subject about 1 minute to about 24 hours apart. For example, acetyl-leucine and 4-AP or acetazolamide are administered about 1 minute apart, 5 minutes apart, 10 minutes apart, 30 minutes apart, 45 minutes apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, or 11 hours to 12 hours apart.

“Subject” means a human.

“Subject in need thereof” means a human who has ataxia and is need of treatment.

A “therapeutically effective amount” of acetyl-leucine or 4-AP or acetazolamide is any amount of each active agent which, when administered to a subject, is the amount that is needed to produce the desired effect, which, for the present disclosure, can be therapeutic and/or prophylactic. The dose may be determined according to various parameters, such as the specific active agent used; the age, weight and condition of the patient to be treated; the route of administration; and the required regimen. A physician will be able to determine the required route of administration and dosage for any particular patient. For example, a daily dose of each active agent may be from about 0.1 to about 225 mg per kg, from about 1 to about 150 mg per kg, or from about 10 to about 100 mg per kg of body weight.

As used herein, “treating” or “treatment” refers to any indicia of success in preventing, arresting, or ameliorating a disease, disorder, condition, or syndrome, e.g., ataxia, in a subject, and/or preventing, arresting, or ameliorating any one or more symptoms a disease, disorder, condition, or syndrome in a subject, including any objective or subjective parameter such as abatement; remission; preventing, diminishing, inhibiting, or eliminating one or more symptoms, e.g., migraine; making the disease, disorder, condition, or syndrome more tolerable to the subject; slowing in the worsening of the disease, disorder, condition, or syndrome; or improving the physical or mental well-being of the subject in need thereof.

The terms “treating” or “treatment” also encompasses inducing inhibition, regression, or stasis of the disease, disorder, condition, or syndrome. For example, treatment of a subject in need of treatment for ataxia includes reducing the bouts of ataxia in the subject, inducing clinical response, inhibiting or reducing progression of the episodic ataxia, or inhibiting or reducing one or more complications of the ataxia.

Preventing, arresting, or ameliorating an injury or pathology of a disease, disorder, condition, or syndrome, such as preventing, diminishing, inhibiting, or eliminating one or more symptoms of disease, disorder, condition, or syndrome can be based on objective and/or subjective parameters, including, e.g., the results of physical examination(s), neurological examination(s), and/or psychiatric evaluation(s). The success of treatment for certain diseases e.g., ataxia, e.g., EA, may be measured or evaluated by, for example, comparing the severity of the disease, e.g., objective and/or subjective parameters of the ataxia, before treatment with acetyl-leucine and 4-AP or acetazolamide is initiated, with the severity of the disease following the initiation of treatment with acetyl-leucine and 4-AP or acetazolamide. For example, the severity of ataxia may be assessed using a scale, index, rating, or score. In one embodiment, the treatment described herein improves such an assessment from a value or degree characteristic of a symptomatic subject to a value or degree characteristic of a non-symptomatic subject. In one embodiment, the treatment described herein improves such an assessment compared to a baseline. The baseline may be, for example, the subject's condition before initiating any treatment for the disease or before initiating treatment for the disease with acetyl-leucine and 4-AP or acetazolamide. Alternatively, the baseline may be, for example, the subject's condition after a certain time period on treatment for the disease. In one embodiment, treatment with acetyl-leucine and 4-AP or acetazolamide as described herein improves the subject's assessment (e.g., scale, index, rating, or score of objective and/or subjective parameters, e.g., SARA, compared to a baseline by at least 10%, at least 20%, at least 30%, at least 40%, or at least 50%. In one embodiment, assessment is improved by at least 60%, at least 70%, at least 80%, at least 90%, or 100%.

In one embodiment, acetyl-leucine is administered at a dose ranging from about 500 mg to about 30 g per day. For example acetyl-leucine is administered at a dose ranging from about 500 mg to about 15 g per day, a dose ranging from about 1.5 g to about 10 g per day, optionally by solid oral or liquid oral route. N-Acetyl-DL-leucine, may be administered, for example, in a dose according to that of Tanganil®, which is prescribed to adults in a dose of 1.5 g to 2 g per day, 3-4 tablets in two doses, morning and evening.

If a single enantiomer of acetyl-leucine, i.e., N-acetyl-D-leucine or N-acetyl-L-leucine, is administered the doses may be reduced accordingly. For instance, if only N-acetyl-L-leucine or if only N-acetyl-D-leucine is administered, the dose may range from about 250 mg to about 15 g per day, range from about 250 mg to about 10 g per day, or range from about 250 mg to about 5 g per day, such as from about 0.75 g to about 5 g per day.

In one embodiment, the administered dose ranges of acetyl-leucine are from about 1 g to about 30 g per day. For example, the administered dose ranges of acetyl-leucine are from about 1 g to about 15 g per day, from about 1 g to about 10 g per day, or from about 1.5 g to about 7 g per day, from 15.1 g to about 30 g per day, 16 g to about 30 g per day, 17 g to about 30 g per day, 18 g to about 30 g per day, 19 g to about 30 g per day, or 20 g to about 30 g per day. It may be from about 2, 3, 4, 5, 6, 7, 8, 9, 10, 11, 12, 13, or 14 g to about 15 g per day. It may be from about 2, 3, 4, 5, 6, 7, 8 or 9 g to about 10 g per day. It may be from 15.1, 16, 17, 18, 19, 20, 21, 22, 23, 24, 25, 25, 27, 28, or 29 g to about 30 g per day. It may be more than about 1.5 g per day, but less than about 15, 14, 13, 12, 11, 10, 9, 8, 7, 6 or 5 g per day. In one embodiment, the dose ranges from about 4 g to about 6 g per day. In one embodiment, the dose ranges from about 4 g to about 5 g per day. In one embodiment, the dose is about 4.5 g per day. In one embodiment, the dose is about 5 g per day. In one embodiment, the dose is about 1 g per day, about 2 g per day, about 3 g per day, about 4 g per day, about 5 g per day, about 6 g per day, about 7 g per day, about 8 g per day, about 9 g per day, about 10 g per day, about 11 g per day, about 12 g per day, about 13 g per day, about 14 g per day, or about 15 g per day. In another embodiment, the dose is about 16 g per day, about 17 g per day, about 18 g per day, about 19 g per day, or about 20 g per day. In another embodiment, the dose is about 21 g per day, about 22 g per day, about 23 g per day, about 24 g per day, about 25 g per day, about 26 g per day, about 27 g per day, about 28 g per day, about 29 g per day, or about 30 g per day. In one embodiment, these doses are administered in a solid oral dosage form, notably tablets. In another embodiment, these doses are for acetyl-leucine when in its racemic form. Doses for acetyl-leucine when an enantiomeric excess is present may be lower, for example, around 50% lower. The above recited dose-ranges when halved are thus also explicitly encompassed by the present disclosure.

In one embodiment, 4-AP is administered at a dose ranging from about 0.01 mg to about 1 g per day, e.g., about 5 mg to about 100 mg per day, e.g., about 15 mg to about 30 mg per day. In another embodiment, 4-AP is administered at a dose of about 5 mg per day. In another embodiment, 4-AP is administered at a dose of about 10 mg per day. In another embodiment, 4-AP is administered at a dose of about 15 mg per day. In another embodiment, 4-AP is administered at a dose of about 20 mg per day. In another embodiment, 4-AP is administered at a dose of about 25 mg per day. In another embodiment, 4-AP is administered at a dose of about 30 mg per day. In another embodiment, 4-AP is administered in two divided doses.

In one embodiment, acetazolamide is administered at a dose ranging from about 100 mg to about 2000 mg per day. In another embodiment, acetazolamide is administered at a dose of about 250 mg per day. In another embodiment, acetazolamide is administered at a dose of about 500 mg per day. In another embodiment, acetazolamide is administered at a dose of about 750 mg per day. In another embodiment, acetazolamide is administered at a dose of about 1000 mg per day.

In one embodiment, the total daily dose of acetyl-leucine or 4-AP or acetazolamide may be spread across multiple administrations, i.e., administration may occur two or more times a day to achieve the total daily dose. As an example, the required number of tablets to provide the total daily dose of a acetyl-leucine may be split across two administrations (for example, in the morning and evening) or three administrations (for example, in the morning, noon and evening). Each dose may be suitably administered with or without food. For example, N-acetyl-L-leucine or N-acetyl-DL-leucine may be dosed by about 1 or about 2 hours before meals, such as at least about 20 minutes, at least about 30 minutes, at least about 40 minutes, or at least about 1 hour before meals, or may be dosed by about 1, about 2, or about 3 hours after meals, such as waiting at least about 20 minutes, at least about 30 minutes, at least about 1 hour, at least about 1.5 hours, at least about 2 hours, or at least about 2.5 hours after meals. For example, a total daily dose of 4.5 g acetyl-DL-leucine may be administered as three Tanganil® (or equivalent) tablets before, with, or after breakfast, three further tablets before, with, or after lunch and three further tablets before, with, or after dinner.

The treatment duration for the combination of acetyl-leucine and 4-AP or acetazolamide may be about seven days or more. For example, the treatment duration may be about two weeks or more, about three weeks or more, about one month or more, about six weeks or more, about seven weeks or more, or about two months or more. In one embodiment, the treatment duration is about three months or more, e.g., about four months or more, about five months or more or about six months or more. The treatment duration may also be about 1 year or more, about 2 years or more, about 4 years or more, about 5 years or more, or about 10 years or more. The treatment duration may also be the life-time of the subject.

Any and all combinations of dosage form, dose amount, dosing schedule, and treatment duration for the combination of acetyl-leucine and 4-AP or acetazolamide are envisaged and encompassed by the disclosure. In one embodiment, the dose of acetyl-leucine is from about 4 g to about 10 g per day, taken across one, two, or three administrations per day, for a treatment duration of about two months or more. In another embodiment, the dose of acetyl-leucine is more than 4 g but no more than 5 g per day, taken across one, two, or three administrations per day, for a treatment duration of about six months or more. The dosage form may be a solid oral dosage form, notably tablets.

In one embodiment, the combination of acetyl-leucine and 4-AP or acetazolamide is used for treating ataxia or one or more symptoms of ataxia. In another embodiment, the ataxia is EA. In another embodiment, the EA is EA2. As used herein, “treating an ataxia or one or more symptoms of an ataxia” and the like refer to delaying onset of ataxia or one or more symptoms of ataxia that would otherwise be expected to manifest according to typical disease progression, reducing the severity of ataxia or reducing the severity of or eliminating one or more existing symptoms associated with ataxia, delaying progression of ataxia or one or more symptoms of ataxia over time as compared to typical disease progression, and/or reversing progression of ataxia or one or more symptoms of ataxia over time.

A “symptom” of ataxia includes any clinical or laboratory manifestation associated with ataxia, e.g., poorly coordinated gait and finger/hand movements, dysarthria, nystagmus, etc., and is not limited to what the subject can feel or observe. For example, symptoms of EA include, but are not limited to, vertigo, dysarthria, diplopia, weakness, tonic upward gaze, headache, seizure, dystonia, and/or cognitive impairment. Onset of symptoms may range from birth to adulthood.

Progression of ataxia or one or more symptoms of ataxia over time or through treatment can be monitored, for example, using one or more known tests at two or more time points and comparing the results. Disease progression and/or severity can be assessed, for example, using the Scale for the Assessment and Rating of Ataxia (SARA), Spinocerebellar Ataxia Functional Index (SCAFI), the International Cooperative Ataxia Rating Scale (ICARS), the brief ataxia rating scale (BARS), the modified Disability Rating Scale (mDRS), EuroQol 5Q-5D-5L (EQ-5D-5L), the visual analogue scale (VAS), Wechsler Adult Intelligence Scale-Revised (WAIS-R), Wechsler Intelligence Scale for Children-IV (WISC-IV), Montreal Cognitive Assessment (MoCA), or other suitable tests.

### EXAMPLES

### Example 1

A 47-year old German female presented with recurrent episodic attacks of postural imbalance (no vertigo sensu stricto, no nausea or vomiting) with the feeling of falling forward, and an associated holocephalic headache that began around the age of 15. The attacks occurred daily, most often in stressful situations and usually lasted for several hours. Other triggers were not evident. By the time the patient presented, she felt permanently dizzy and posturally insecure in between the attacks, rendering her dependent on regular help in her daily routine. There was no evidence form the patient history for seizures or syncope. Diagnosis had remained unknown and no specific treatment had tried before.

The patient's clinical and neuro-ophthalmological examination revealed a cerebellar ocular motor dysfunction with bilateral horizontal gaze-evoked and rebound-nystagmus, saccadic pursuit in all directions, hypermetric horizontal saccades, reduced optokinetic nystagmus in all directions, and horizontally reduced VOR bilaterally. Finger-to-finger-following showed slightly hypermetric movements. Romberg's test revealed an imbalance in tandem stance. Quantitative gait analysis with the GAITRite system showed slightly reduced velocity and step length but no further aberrance in her age cohort. Her cranial MM showed several small supratentorial white matter lesions but no distinct vermian atrophy as is sometimes reported in EA2.

Genetic testing using “Next Generation Sequencing and Sanger Sequencing” methods revealed a novel, heterozygous, pathogenic variant in exon 16 of the CACNA1A gene NM_001127221.1: (c2070_2071delinsGGAG, p.(Phe690Leufs*9)). Deletions of cytosine and thymidine at position 2070-2071 and insertion of four nucleotides at this position in exon 16 of the CACNA1A gene were identified. This leads to a frameshift during translation and to an early stop of protein synthesis at codon position 698 after the incorporation of eight changed amino acids.

In three other family members (FIG. 1) of the patient, the same heterozygous CACNA1A mutation was found. The clinical phenotypes varied considerably: The 68-year old mother of the patient suffered from a slowly progressive imbalance of gait, first recognized at the age of 40, but no ataxic episodes, and was still living unaided. The 45-year old brother of our patient did not report any worsening symptoms or episodes of ataxia, vertigo or imbalance. He only reported to have been suffering from intermittent headache since early childhood, which was partly responsive to ibuprofen. The 15-year old nephew of our patient (her brother's son) has suffered from episodic attacks with imbalance of gait, vertigo, and headache, typically lasting for several hours and triggered by stress since the age of 14.

One reason for this heterogeneous phenotype might be incomplete penetrance. There is evidence of intra-familial incomplete penetrance due to a pathogenic CACNA1A variant in exon 6 in the literature. Angelini et al., European Journal of Medical Genetics 62:103530. doi:10.1016/j.ejmg.2018.08.011 (2019). But an involvement of other genes cannot be excluded. One of the major limiting methodological factors is no general certainty of covering a genetic mosaic.

The patient did not respond to treatment with 4-aminopyridine (20 mg/d) or acetazolamide (250 mg-500 mg/d). Griggs et al., Neurology 28:1259-1264. doi:10.1212/wn1.28.12.1259 (1978). Acetazolamide also caused paresthesia and kidney dysfunction as side effects that the patient did not tolerate and therefore the dosage was not further increased.

Next, the patient was treated with the combination of 4-aminopyridine (Fampyra™ (15 mg/d) and acetyl-DL-leucine (5 g/d). Over the ensuing observation period of 12 months a surprising stabilization of objective clinical and functional measures (such as videooculography, gait analysis and ataxia scores (SARA score 4)) as well as a subjectively relevant improvement of the patient's daily routine was observed interictally. No further disease progression has been observed.

### Example 2

The patient is a 26 year old male from Slovakia. Physical exertion and stress triggered vertigo and vomiting starting at the age of 8. By the age of 17, the patient experienced frequent vomiting, vertigo, ataxia, and incomprehensible speech. The patient was diagnosed with episodic ataxia type 2. Without medication, the patient experienced one or two episodes of ataxia per day.

At age 18, the patient was treated with acetazolamide. The frequency of the episodes decreased to two or three times per week for a period of time, and then returned to a frequency of one episode of ataxia per day.

The patient was treated with the combination of acetalozamide and 5 g per day of tanganil (acetyl-DL-leucine), and his condition stabilized. After 3 months of treatment, the patient has experienced one episode of ataxia and two attacks of vertigo. The patient also takes sertraline. Genetic testing showed the patient had CACNA1A variant p.Gly297Arg (c.889G>A).

### REFERENCES

1. Kipfer S, Strupp M (2014) The Clinical Spectrum of Autosomal-Dominant Episodic Ataxias. Movement disorders clinical practice 1 (4):285-290. doi:10.1002/mdc3.12075
2. Jen J C, Wan J (2018) Episodic ataxias. Handbook of clinical neurology 155:205-215. doi:10.1016/b978-0-444-64189-2.00013-5
3. Imbrici P, Eunson L H, Graves T D, Bhatia K P, Wadia N H, Kullmann D M, Hanna M G (2005) Late-onset episodic ataxia type 2 due to an in-frame insertion in CACNA1A. Neurology 65 (6):944-946. doi:10.1212/01.wn1.0000176069.64200.28
4. Jen J, Kim G W, Baloh R W (2004) Clinical spectrum of episodic ataxia type 2. Neurology 62 (1):17-22. doi:10.1212/01.wn1.0000101675.61074.50
5. Riant F, Vahedi K, Tournier-Lasserve E (2011) [Hereditary episodic ataxia]. Revue neurologique 167 (5):401-407. doi:10.1016/j.neurol.2010.10.016
6. Ophoff R A, Terwindt G M, Vergouwe M N, van Eijk R, Oefner P J, Hoffman S M, Lamerdin J E, Mohrenweiser H W, Bulman D E, Ferrari M, Haan J, Lindhout D, van Ommen G J, Hofker M R, Ferrari M D, Frants R R (1996) Familial hemiplegic migraine and episodic ataxia type-2 are caused by mutations in the Ca2+ channel gene CACNL1A4. Cell 87 (3):543-552. doi:10.1016/s0092-8674(00)81373-2
7. Jen J C, Graves T D, Hess E J, Hanna M G, Griggs R C, Baloh R W (2007) Primary episodic ataxias: diagnosis, pathogenesis and treatment. Brain 130 (Pt 10):2484-2493. doi:10.1093/brain/awm126
8. Isaacs D A, Bradshaw M J, Brown K, Hedera P (2017) Case report of novel CACNA1A gene mutation causing episodic ataxia type 2. SAGE open medical case reports 5:2050313x17706044. doi:10.1177/2050313x17706044
9. Guterman E L, Yurgionas B, Nelson A B (2016) Pearls & Oy-sters: Episodic ataxia type 2: Case report and review of the literature. Neurology 86 (23):e239-241. doi:10.1212/wn1.0000000000002743
10. Richards S, Aziz N, Bale S, Bick D, Das S, Gastier-Foster J, Grody W W, Hegde M, Lyon E, Spector E, Voelkerding K, Rehm H L (2015) Standards and guidelines for the interpretation of sequence variants: a joint consensus recommendation of the American College of Medical Genetics and Genomics and the Association for Molecular Pathology. 17 (5):405-424. doi:10.1038/gim.2015.30
11. Mantuano E, Romano S, Veneziano L, Gellera C, Castellotti B, Caimi S, Testa D, Estienne M, Zorzi G, Bugiani M, Rajabally Y A, Barcina M J, Servidei S, Panico A, Frontali M, Mariotti C (2010) Identification of novel and recurrent CACNA1A gene mutations in fifteen patients with episodic ataxia type 2. Journal of the neurological sciences 291 (1-2):30-36. doi:10.1016/j.jns.2010.01.010
12. Maksemous N, Roy B, Smith R A, Griffiths L R (2016) Next-generation sequencing identifies novel CACNA1A gene mutations in episodic ataxia type 2. Molecular genetics & genomic medicine 4 (2):211-222. doi:10.1002/mgg3.196
13. Angelini C, Van Gils J, Bigourdan A, Jouk P S, Lacombe D, Menegon P, Moutton S, Riant F, Sole G, Tournier-Lasserve E, Trimouille A, Vincent M, Goizet C (2019) Major intra-familial phenotypic heterogeneity and incomplete penetrance due to a CACNA1A pathogenic variant. European journal of medical genetics 62 (6):103530. doi:10.1016/j.ejmg.2018.08.011
14. Ilg W, Bastian A J, Boesch S, Burciu R G, Celnik P, Claassen J, Feil K, Kalla R, Miyai I, Nachbauer W, Schols L, Strupp M, Synofzik M, Teufel J, Timmann D (2014) Consensus paper: management of degenerative cerebellar disorders. Cerebellum (London, England) 13 (2):248-268. doi:10.1007/s12311-013-0531-6
15. Gandini J, Manto M (2020) The neurological update: therapies for cerebellar ataxias in 2020. doi:10.1007/s00415-020-09717-3
16. Strupp M, Kalla R, Dichgans M, Freilinger T, Glasauer S, Brandt T (2004) Treatment of episodic ataxia type 2 with the potassium channel blocker 4-aminopyridine. Neurology 62 (9):1623-1625. doi:10.1212/01.wn1.0000125691.74109.53
17. Strupp M, Kalla R, Claassen J, Adrion C, Mansmann U, Klopstock T, Freilinger T, Neugebauer H, Spiegel R, Dichgans M, Lehmann-Horn F, Jurkat-Rott K, Brandt T, Jen J C, Jahn K (2011) A randomized trial of 4-aminopyridine in EA2 and related familial episodic ataxias. Neurology 77 (3):269-275. doi:10.1212/WNL.0b013e318225ab07
18. Griggs R C, Moxley R T, 3rd, Lafrance R A, McQuillen J (1978) Hereditary paroxysmal ataxia: response to acetazolamide. Neurology 28 (12):1259-1264. doi:10.1212/wn1.28.12.1259
19. Kalla R, Strupp M (2019) Aminopyridines and Acetyl-D L-leucine: New Therapies in Cerebellar Disorders. Current neuropharmacology 17 (1):7-13. doi:10.2174/1570159x16666180905093535

All of the features described herein (including any accompanying claims, abstract and drawings), and/or all of the steps of any method so disclosed, may be combined with any of the above aspects in any combination, except combinations where at least some of such features and/or steps are mutually exclusive.

It is to be understood that the foregoing embodiments and exemplifications are not intended to be limiting in any respect to the scope of the disclosure, and that the claims presented herein are intended to encompass all embodiments and exemplifications whether or not explicitly presented herein.

All patents, patent applications, and publications cited herein are fully incorporated by reference in their entirety.

1. A method of treating ataxia in a subject in need thereof, the method comprising administering a combination of a therapeutically effective amount of acetyl-leucine and (i) a therapeutically effective amount of 4-aminopyridine or (ii) a therapeutically effective amount of acetazolamide to the subject.

2. The method of claim 1, wherein acetyl-leucine and 4-aminopyridine or acetazolamide are administered simultaneously.

3. The method of claim 2, wherein acetyl-leucine and 4-aminopyridine or acetazolamide are administered as a single pharmaceutical formulation.

4. (canceled)

5. The method of claim 1, wherein acetyl-leucine and 4-aminopyridine or acetazolamide are administered sequentially.

6. The method of claim 5, wherein acetyl-leucine is administered before 4-aminopyridine or acetazolamide.

7. The method of claim 5, wherein acetyl-leucine is administered after 4-aminopyridine or acetazolamide.

8. The method of claim 5, wherein acetyl-leucine and 4-aminopyridine or acetazolamide are administered about 1 minute to about 6 hours apart.

9. (canceled)

10. (canceled)

11. (canceled)

12. The method of claim 1, wherein acetyl-leucine is administered once, twice, or three times per day.

13. The method of claim 1, wherein 4-aminopyridine or acetazolamide is administered once, twice, or three times per day.

14. The method of claim 1, wherein about 3 g to about 15 g of acetyl-leucine is administered per day.

15. The method of claim 1, wherein about 10 mg to about 30 mg of 4-aminopyridine or about 500 mg to about 1000 mg of acetazolamide is administered per day.

16. The method of claim 1, wherein acetyl-leucine and 4-aminopyridine or acetazolamide are administered as first-line therapy to treat the episodic ataxia.

17. The method of claim 1, wherein the ataxia is episodic ataxia.

18. The method of claim 17, wherein the episodic ataxia is episodic ataxia type 2.

19. The method of claim 1, wherein acetyl-leucine is administered in combination with 4-aminopyridine to the subject.

20. The method of claim 1, wherein acetyl-leucine is administered in combination with acetazolamide to the subject.

21. The method of claim 1, wherein the acetyl-leucine is N-acetyl-DL-leucine.

22. The method of claim 1, wherein the acetyl-leucine is N-acetyl-L-leucine.

23. The method of claim 1, wherein the subject has a deletion of cytosine and thymidine at position 2070-2071 in exon 16 of the CACNA1A gene.

24. A kit comprising acetyl-leucine and 4-aminopyridine or acetazolamide for treating ataxia in a subject.

25. (canceled)

26. (canceled)
